(19) United States
(12) Patent Application Publication
(10) Pub. No.: US 2009/0042825 A1
(43) Pub. Date: Feb. 12, 2009

(54) Composition, method of preparation & application of concentrated formulations of condensed nucleic acids with a cationic lipopolymer

(76) Inventors: Majed Matar, Madison, AL (US); Jason Fewell, Madison, AL (US); Danny H. Lewis, Hartselle, AL (US); Khursheed Anwer, Madison, AL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/890,805

(22) Filed: Aug. 6, 2007

(51) Int. Cl. A61K 31/70; A61P 43/00

(52) U.S. Cl. 514/44

(57) ABSTRACT

Compositions, methods, and applications that increase the efficiency of nucleic acid transfection are provided. In one aspect, a pharmaceutical composition may include at least about 0.5 mg/ml concentration of a nucleic acid condensed with a cationic lipopolymer suspended in an isotonic solution, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The composition further may include a filler excipient.

### FIELD OF THE INVENTION

The present invention relates to compositions, methods of preparation, and applications of concentrated and stable formulations of nucleic acids with a cationic lipopolymer. Accordingly, this invention involves the fields of molecular biology and biochemistry.

### BACKGROUND OF THE INVENTION

Synthetic gene delivery vectors have considerable advantage over viral vectors due to better safety compliance, simple chemistry, and cost-effective manufacturing. However, due to low transfection efficiency of the synthetic vectors as compared to that of the viral vectors, most of the development in synthetic gene delivery systems has focused on improving delivery efficiency. Consequently, little attention has been given to the pharmaceutical development of synthetic delivery systems, although problems have been identified in formulation stability, scale up, and dosing flexibility. For example, aqueous suspensions of DNA complexes with synthetic vectors appear to be generally unstable and aggregate over time, especially at concentrations required for optimal dosing in a clinical setting. This physical instability is believed to be one of the underlying reasons for loss of transfection activity. Manifestation of particle rupture or fusion due to high curvature of the lipid bilayer or physical dissociation of lipid from DNA have also been postulated as potential underlying reasons for poor stability and aggregation of cationic lipid based gene delivery complexes. Chemical modification such as oxidative hydrolysis of the delivery vectors may also contribute to particle instability.

Because of poor stability, the early clinical trials required that DNA formulations be prepared by the bedside. Not having the ability to prepare and store the clinical product at concentrations required for optimal dosing is a major obstacle in the broad clinical practice and commercialization of the non-viral DNA products. This would require physicians training on drug formulation and pose on-site quality control measures.

Freeze-drying is a useful method for improving long-term stability of a number of drug pharmaceuticals. However, this process may not be suitable for drying prior DNA complexes with synthetic vectors as it may alter their physicochemical properties and lead to aggregation and loss of transfection. Increase in physical contact among the DNA particles as the formulation is gradually concentrated during the lyophilization process may promote particle aggregation. Several approaches have been attempted to prevent formulation aggregation and damage during lyophilization. These include the addition of low molecular weight sugars, dextrans, and polyethylene glycol. Addition of sugars is often the most commonly used approach for this purpose. Many of the test sugars have been found to prevent formulation damage and particle aggregation to some extent, but the quality of this effect varies with the type of sugar and the delivery vector used.

Although lyophilization provides some improvement in formulation shelf life, the conditions required to produce lyophilized DNA products allow for only limited pharmaceutical applications. Even with the most effective lyoprotectant sugars, a very high sugar/DNA molar ratio is required for stability. As a result, the lyophilized product often must be diluted by a very large factor to obtain an isotonic formulation, which results in a drop in the final DNA concentration to ≦0.1 mg/ml. Although low concentration formulations are sufficient for in vitro studies, their clinical application may be limited due to high volume requirement for optimal dosing. For example, at the optimal sugar concentration needed for stability, a 1 mg dose of DNA may need to be diluted in 5-10 ml to maintain isotonicity, which is too large a volume for local in vivo administration. This pharmaceutical limitation, prohibitive of flexible dosing, is one of the principal contributors to suboptimal efficacy of synthetic gene delivery systems in human clinical trials and warrants the need for more concentrated DNA formulations that are stable and biologically active.

### SUMMARY OF THE INVENTION

Accordingly, the present invention provides compositions, methods, and applications that increase the efficiency and dosing flexibility of nucleic acid transfection. In one aspect, for example, a pharmaceutical composition is provided, including at least about 0.5 mg/ml concentration of a nucleic acid condensed with a cationic lipopolymer suspended in an isotonic solution, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The composition further may include a filler excipient. In another aspect, the concentration of the nucleic acid may be at least about 1 mg/ml. In yet another aspect, the concentration of the nucleic acid may be at least about 3 mg/ml. In a further aspect, the concentration of the nucleic acid may be at least about 5 mg/ml. In yet a further aspect, the concentration of the nucleic acid may be at least about 10 mg/ml.

The nucleic acid may be fully or partially condensed, in some cases depending on the compositional makeup of the nucleic acid and the conditions under which condensation occurs. In one aspect, however, the nucleic acid may be at least about 30% condensed. In another example, the nucleic acid may be at least about 50% condensed. In yet another aspect, the nucleic acid may be at least about 70% condensed. In a further aspect, the nucleic acid may be at least about 90% condensed.

A variety of cationic polymers may be utilized to form the cationic polymer backbones according to aspects of the present invention. Non-limiting examples of cationic polymers may include polyethylenimine (PEI), poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, and combinations thereof. In one specific aspect, for example, the cationic polymer backbone may be polyethylenimine or PEI.

Numerous nucleic acids are suitable for use according to the various aspects of the present invention. For example, in one aspect the nucleic acid may be a plasmid encoding for a peptide. In another aspect, the nucleic acid may be a plasmid encoding for a member selected from the group consisting of interleukin-2, interleukin-4, interleukin-7, interleukin-12, interleukin-15, interferon-α, interferon-β, interferon-γ, colony stimulating factor, granulocyte-macrophage colony stimulating factor, angiogenic agents, clotting factors, hypoglycemic agents, apoptosis factors, anti-angiogenic agents, thymidine kinase, p53, IP10, p16, TNF-α, Fas-ligand, tumor antigens, neuropeptides, viral antigens, bacterial antigens, and combinations thereof. In one specific aspect, for example, the nucleic acid may be a plasmid encoding for interleukin-12. In another specific aspect, the nucleic acid may be a plasmid encoding for an inhibitory ribonucleic acid. In yet another specific aspect, the nucleic acid may be a synthetic short interfering ribonucleic acid. In a further specific aspect, the nucleic acid may be an anti-sense molecule designed to inhibit expression of a therapeutic peptide.

In another aspect, the cationic lipopolymer may include a targeting moiety covalently attached to either the cationic lipopolymer or to the polyethylene glycol molecule. Although any targeting moiety may be utilized, non-limiting examples may include transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents, lysosomotrophic agents, nucleus localization signals (NLS), and combinations thereof.

A wide variety of excipients may be utilized as fillers for inclusion in compositions according various aspects of the present invention. For example, in one aspect non-limiting examples of fillers may generally include sugars, sugar alcohols, starches, celluloses, and combinations thereof. In another aspect, the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, polyethylene glycol, and combinations thereof. In a specific aspect, the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, and combinations thereof. In a more specific aspect, the filler excipient may be sucrose. In another more specific aspect, the filler excipient may be lactose.

The present invention also provides methods of making compositions according to the various aspects of the present invention. In one aspect, for example, a method of making a pharmaceutical composition having a condensed nucleic acid concentrated in an isotonic solution to at least 0.5 mg/ml. Such a method may include mixing a nucleic acid and a cationic lipopolymer in a filler excipient, the cationic lipopolymer including a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The mixture is then lyophilized to a powder. The powder may be reconstituted with a diluent to form a solution including at least about 0.5 mg/ml condensed nucleic acid in an isotonic solution.

Lyophilized compositions are also considered to be within the scope of the present invention. For example, a lyophilized pharmaceutical composition may include a lyophilized mixture of a filler excipient and a nucleic acid condensed with a cationic lipopolymer, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10.

The present invention additionally provides methods for transfecting various cells and tissues. In one aspect, for example, a method of transfecting a mammalian cell is provided, including contacting the mammalian cell with the composition as described above, and incubating the mammalian cell under conditions to allow the composition to enter the cell and elicit biological activity of the nucleic acid. It should be noted that transfection may be accomplished on cell or tissue cultures, or they may be accomplished by contacting cells or tissue within the body of a subject. For example, in one aspect a targeted tissue may be transfected by delivering a composition into a warm blooded organism. Such delivery may be by a form of administration such as intratumoral, intraperitoneal, intravenous, intra-arterial, intratracheal, intrahepaticportal, oral, intracranial, intramuscular, intraarticular and combinations thereof. Additionally, targeted tissue may include any tissue of the subject, including, without limitation, tissue that is localized in an ovary, uterus, stomach, colon, rectum, bone, blood, intestine, pancreas, breast, head, neck, lungs, spleen, liver, kidney, brain, thyroid, prostate, urinary bladder, thyroid, skin, abdominal cavity, thoracic cavity, and combinations thereof.

### DESCRIPTION OF THE DRAWINGS

FIG. 1 shows a diagram of a manufacturing process according to one embodiment of the present invention.

FIG. 2 shows graphs of particle size of nucleic acids in concentrated and non-concentrated states according to another embodiment of the present invention.

FIG. 3 shows results of an electrophoretic experiment to show nucleic acid condensation according to yet another embodiment of the present invention.

FIG. 4 shows graphs of transfection activity according to a further embodiment of the present invention.

FIG. 5 shows photographs of neural slices demonstrating results according to yet a further embodiment of the present invention.

FIG. 6 shows graphs demonstrating anticancer efficacy according to another embodiment of the present invention.

FIG. 7 shows graphs demonstrating particle size of various cationic polymers, some according to one embodiment of the present invention.

FIG. 8 shows graphs demonstrating luciferase expression of nucleic acids associated with various cationic polymers, some according to one embodiment of the present invention.

FIG. 9 shows a graph of the long-term stability of a composition according to yet another embodiment of the present invention.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to a polymer containing “a molecule” includes reference to a polymer having one or more of such molecules, and reference to “an antibody” includes reference to one or more of such antibodies.

Definitions

Before the present composition, its method of preparation and application is disclosed and described, it is to be understood that this invention is not limited to the particular configurations, process steps, and materials disclosed herein as such configurations, process steps, and materials may vary somewhat. It is also to be understood that the terminology employed herein is used for describing particular embodiments only and is not intended to be limiting since the scope of the present invention will be limited only by the appended claims and equivalents thereof.

As used herein, the terms “transfecting” and “transfection” refer to the transportation of nucleic acids from the environment external to a cell to the internal cellular environment, with particular reference to the cytoplasm and/or cell nucleus. Without being bound by any particular theory, it is to be understood that nucleic acids may be delivered to cells either after being encapsulated within or adhering to one or more cationic polymer/nucleic acid complexes or being entrained therewith. Particular transfecting instances deliver a nucleic acid to a cell nucleus.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “composition” refers to a mixture of two or more compounds, elements, or molecules. In some aspects the term “composition” may be used to refer to a mixture of a nucleic acid and a delivery system. In one aspect, such a composition may include nucleic acid condensates of 50-300 nm in size.

As used herein, “N:P ratio” refers to a molar ratio between the amine nitrogen in the functionalized cationic lipopolymer and the phosphate in the nucleic acid.

As used herein, “physicochemical properties” refers to various properties such as, without limitation, particle size and surface charge of nucleic acid complexes with a cationic polymer, pH and osmolarity of the particle solution, etc.

As used herein, the terms “administration,” “administering,” and “delivering” refer to the manner in which a composition is presented to a subject. Administration can be accomplished by various art-known routes such as oral, parenteral, transdermal, inhalation, implantation, etc. Thus, an oral administration can be achieved by swallowing, chewing, sucking of an oral dosage form comprising the composition. Parenteral administration can be achieved by injecting a composition intravenously, intra-arterially, intramuscularly, intraarticularly, intrathecally, intraperitoneally, subcutaneously, etc. Injectables for such use can be prepared in conventional forms, either as a liquid solution or suspension, or in a solid form that is suitable for preparation as a solution or suspension in a liquid prior to injection, or as and emulsion. Additionally, transdermal administration can be accomplished by applying, pasting, rolling, attaching, pouring, pressing, rubbing, etc., of a transdermal composition onto a skin surface. These and additional methods of administration are well-known in the art. In one specific aspect, administration may include delivering a composition to a subject such that the composition circulates systemically and binds to a target cell to be taken up by endocytosis.

As used herein, the term “nucleic acid” refers to DNA and RNA, as well as synthetic congeners thereof. Non-limiting examples of nucleic acids may include plasmid DNA encoding protein or inhibitory RNA producing nucleotide sequences, synthetic sequences of single or double strands, missense, antisense, nonsense, as well as on and off and rate regulatory nucleotides that control protein, peptide, and nucleic acid production. Additionally, nucleic acids may also include, without limitation, genomic DNA, cDNA, siRNA, shRNA, mRNA, tRNA, rRNA, hybrid sequences or synthetic or semi-synthetic sequences, and of natural or artificial origin. In one aspect, a nucleotide sequence may also include those encoding for synthesis or inhibition of a therapeutic protein. Non-limiting examples of such therapeutic proteins may include anti-cancer agents, growth factors, hypoglycemic agents, anti-angiogenic agents, bacterial antigens, viral antigens, tumor antigens or metabolic enzymes. Examples of anti-cancer agents may include interleukin-2, interleukin-4, interleukin-7, interleukin-12, interleukin-15, interferon-α, interferon-β, interferon-γ, colony stimulating factor, granulocyte-macrophage stimulating factor, anti-angiogenic agents, tumor suppressor genes, thymidine kinase, eNOS, iNOS, p53, p16, TNF-α, Fas-ligand, mutated oncogenes, tumor antigens, viral antigens or bacterial antigens. In another aspect, plasmid DNA may encode for an shRNA molecule designed to inhibit protein(s) involved in the growth or maintenance of tumor cells or other hyperproliferative cells. Furthermore, in some aspects a plasmid DNA may simultaneously encode for a therapeutic protein and one or more shRNA. In other aspects a nucleic acid may also be a mixture of plasmid DNA and synthetic RNA, including sense RNA, antisense RNA, ribozymes, etc. In addition, the nucleic acid can be variable in size, ranging from oligonucleotides to chromosomes. These nucleic acids may be of human, animal, vegetable, bacterial, viral, or synthetic origin. They may be obtained by any technique known to a person skilled in the art.

As used herein, the term “concentrated” refers to nucleic acid concentrations higher than pre-condensation value.

As used herein, the term “polymeric backbone” is used to refer to a collection of polymeric backbone molecules having a weight average molecular weight within the designated range. As such, when a molecule such as cholesterol is described as being covalently attached thereto within a range of molar ratios, it should be understood that such a ratio represents an average number of cholesterol molecules attached to the collection of polymeric backbone molecules. For example, if cholesterol is described as being covalently attached to a polymeric backbone at a molar ratio of 0.5, then, on average, one half of the polymeric backbone molecules will have cholesterol attached. As another example, if cholesterol is described as being covalently attached to a polymeric backbone at a molar ratio of 1.0, then, on average, one cholesterol molecule will be attached to each of the polymeric backbone molecules. In reality, however, it should be understood that in this case some polymeric backbone molecules may have no cholesterol molecules attached, while other polymeric backbone molecules may have multiple cholesterol molecules attached, and that it is the average number of attached cholesterol molecules from which the ratio is derived. The same reasoning applies to the molar ratio of polyethylene glycol to the polymeric backbone.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins.

As used herein, the terms “covalent” and “covalently” refer to chemical bonds whereby electrons are shared between pairs of atoms.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The Invention

DNA pharmaceuticals that self-assemble into nanoparticles often exhibit poor stability in liquid suspension and may be difficult to prepare at DNA concentrations >0.3 mg/ml, which limits their commercial applications, especially for local delivery where volume constraints would limit flexible dosing. In some cases, lyophilization of DNA complexes in the presence of cryoprotectant sugars may provide better stability to the product, but such an approach does not appear to improve dosing flexibility. Because a very high sugar:DNA ratio (>1000) must be utilized to protect the DNA complexes during lyophilization, a large volume of diluent must be added to maintain isotonicity of the reconstituted product. The addition of such a large volume of diluent limits the final DNA concentration to the pre-lyophilized DNA concentration. For many cationic carriers the final DNA concentration may typically be about 0.1-0.2 mg/ml.

The present invention provides techniques whereby low concentration nucleic acid compositions (e.g., 0.15 mg/ml) may be highly concentrated without affecting the physico-chemical or biological properties of the nucleic acid or nucleic acid compositions. In one aspect, nucleic acid compositions may be concentrated by 33-fold or more without affecting these properties. These highly concentrated nucleic acid compositions allow for a wide range of dosing regimens in vivo, which have previously been tremendously challenging due to poor stability issues associated with prior attempts to achieve concentrations above ˜0.3 mg/ml.

More specifically, the present invention provides concentrated and stable pharmaceutical compositions, including methods for preparing and using such compositions. In one aspect, for example, a pharmaceutical composition is provided including at least about 0.5 mg/ml concentration of a nucleic acid condensed with a cationic lipopolymer suspended in an isotonic solution, the cationic lipopolymer including a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. In another aspect, the molar ratio of polyethylene glycol to cationic polymer backbone in the cationic lipopolymer may be within a range of from about 1 to about 10. In yet another aspect, the molar ratio of polyethylene glycol to cationic polymer backbone in the cationic lipopolymer may be within a range of from about 1 to about 5. In a further aspect, a molar ratio of cholesterol to cationic polymer backbone in the cationic lipopolymer may be within a range of from about 0.3 to about 5. In yet a further aspect, the molar ratio of cholesterol to cationic polymer backbone in the cationic lipopolymer may be within a range of from about 0.4 to about 1.5. Furthermore, the composition includes a filler excipient and may be used for delivery of the nucleic acid to a target cell to elicit, inhibit, or modify a biological response depending on the function of the nucleic acid.

In one aspect, the cholesterol and polyethylene glycol molecules may be independently covalently attached to the cationic polymer backbone, or in other words, the cholesterol and polyethylene glycol molecules may be each covalently attached to the cationic polymer backbone independent of each other. In another aspect, the cholesterol and polyethylene glycol molecules may be dependently covalently attached to the cationic polymer backbone. For example, in one specific aspect, the cholesterol molecule may be coupled to the polyethylene glycol molecule, which is in turn covalently attached to the cationic polymer backbone. The cholesterol molecule may be directly covalently attached to the polyethylene glycol molecule, or indirectly covalently attached via a linker or spacer.

Additionally, in some aspects nucleic acids that have previously been condensed using a secondary condensing system may be further concentrated using the techniques presented herein to achieve greater stability of the complexes at high nucleic acid concentrations. As such, prior to condensation according to aspects of the present invention, the nucleic acid may be in a partially condensed or a non-condensed form. The secondary condensing system may include any condensing material or technique known to one of ordinary skill in the art, including, but not limited to, cationic lipids, cationic peptides, cyclodextrins, cationized gelatin, dendrimers, chitosan, and combinations thereof.

Numerous degrees of condensation of a nucleic acid may be concentrated according to aspects of the present invention. In one aspect, for example, the nucleic acid may fully or substantially fully condensed. In another aspect, the nucleic acid may be at least about 30% condensed. In yet another aspect, the nucleic acid may be at least about 50% condensed. In a further aspect, the nucleic acid may be at least about 70% condensed. In yet a further aspect, the nucleic acid may be at least about 90% condensed.

Additionally, the concentration of nucleic acid in the composition may vary depending on the materials used in the composition, the methods of concentration, and the intended use of the nucleic acid. In one aspect, however, the concentration of the nucleic acid may be at least about 0.5 mg/ml. In another aspect, the concentration of the nucleic acid may be at least about 1 mg/ml. In yet another aspect, the concentration of the nucleic acid may be at least about 3 mg/ml. In a further aspect, the concentration of the nucleic acid may be at least about 5 mg/ml. In yet a further aspect, the concentration of the nucleic acid may be at least about 10 mg/ml. In another aspect, the concentration of the nucleic acid may be at least about 20 mg/ml. In yet another aspect, the concentration of the nucleic acid may be from about 10 mg/ml to about 40 mg/ml.

Various methods may be utilized to determine the degree of condensation of a nucleic acid composition. For example, in one aspect a condensed nucleic acid may be electrophoresed to determine the condensation degree. The electrostatic attraction of the negatively charged nucleic acid to the positively charged cationic lipopolymer inhibits the nucleic acid from moving through an agarose gel. Accordingly, following electrophoresis, condensed nucleic acids will remain immobile in the gel, while non-condensed nucleic acids will have traveled a distance relative to the strength of the electrical current in the gel.

Any known nucleic acid may be utilized in the compositions and methods according to aspects of the present invention, including those examples described above. As such, the nucleic acids described herein should not be seen as limiting. In one aspect, for example, the nucleic acid may include a plasmid encoding for a protein, polypeptide, or peptide. Numerous peptides are well known that would prove beneficial when formulated as pharmaceutical compositions according to aspects of the present invention. Non-limiting examples of a few of such peptides may include interleukin-2, interleukin-4, interleukin-7, interleukin-12, interleukin-15, interferon-α, interferon-β, interferon-γ, colony stimulating factor, granulocyte-macrophage colony stimulating factor, angiogenic agents, clotting factors, hypoglycemic agents, apoptosis factors, anti-angiogenic agents, thymidine kinase, p53, IP10, p16, TNF-α, Fas-ligand, tumor antigens, neuropeptides, viral antigens, bacterial antigens, and combinations thereof. In one specific aspect, the nucleic acid may be a plasmid encoding for interleukin-12. In another aspect, the nucleic acid may be a plasmid encoding for an inhibitory ribonucleic acid. In yet another aspect, the nucleic acid may be a synthetic short interfering ribonucleic acid. In a further aspect, the nucleic acid is an anti-sense molecule designed to inhibit expression of a therapeutic peptide.

As has been described, a cationic lipopolymer may include a cationic polymer backbone having cholesterol and polyethylene glycol covalently attached thereto. The cationic polymer backbone may include any cationic polymer known to one of ordinary skill in the art that may be used to condense and concentrate a nucleic acid according to the various aspects of the present invention. In one aspect, however, the cationic polymer backbone may include polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, and combinations thereof. In one specific aspect, the cationic polymer backbone may be polyethylenimine.

Additionally, the molecular weight of a cationic polymer backbone may vary, depending on numerous factors including the properties of the nucleic acid, the intended use of the composition, etc. In one aspect, however, the cationic polymer backbone may have a molecular weight of from about 100 to about 500,000 Daltons. Furthermore, the molecular weight of the other various components of the cationic lipopolymer may also vary. In one aspect, for example, polyethylene glycol may have a molecular weight of from about 50 to about 20,000 Daltons.

In constructing the pharmaceutical compositions of the present invention, it has been discovered that the molar ratio between the amine nitrogen in the functionalized cationic lipopolymer and the phosphate in the nucleic acid (N:P ratio) may affect the degree to which the nucleic acid may be condensed and/or concentrated. Although the optimal N:P ratio may vary somewhat depending on the chemical characteristics of the nucleic acid, in one aspect the ratio of amine nitrogen in the cationic polymer backbone to phosphate in the nucleic acid is from about 0.1:1 to about 100:1. In another aspect, the ratio of amine nitrogen in the cationic polymer backbone to phosphate in the nucleic acid is from about 3:1 to about 20:1. In yet another aspect, the ratio of amine nitrogen in the cationic polymer backbone to phosphate in the nucleic acid is from about 6:1 to about 15:1. In one specific aspect, the ratio of amine nitrogen in the cationic polymer backbone to phosphate in the nucleic acid is about 11:1.

It is also contemplated that a filler excipient be included in the pharmaceutical composition. Such filler may provide a variety of beneficial properties to the formulation, such as cryoprotection during lyophilization and reconstitution, binding, isotonic balance, stabilization, etc. It should be understood that the filler material may vary between compositions, and the particular filler used should not be seen as limiting. In one aspect, for example, the filler excipient may include various sugars, sugar alcohols, starches, celluloses, and combinations thereof. In another aspect, the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, polyethylene glycol, and combinations thereof. In yet another aspect the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, and combinations thereof. In one specific aspect, the filler excipient may include sucrose. In another specific aspect, the filler excipient may include lactose.

In some aspects it may be beneficial to functionalize the cationic lipopolymer to allow targeting of specific cells or tissues in a subject or culture. Such targeting is well known, and the examples described herein should not be seen as limiting. In one aspect, for example, the cationic lipopolymer may include a targeting moiety covalently attached to either the cationic lipopolymer or to the polyethylene glycol molecule. Such a targeting moiety may allow the cationic lipopolymer to circulate systemically in a subject to locate and specifically target a certain cell type or tissue. Examples of such targeting moieties may include transferrin, asialoglycoprotein, antibodies, antibody fragments, low density lipoproteins, cell receptors, growth factor receptors, cytokine receptors, folate, transferrin, insulin, asialoorosomucoid, mannose-6-phosphate, mannose, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents such as polymixin B and hemaglutinin HA2, lysosomotrophic agents, nucleus localization signals (NLS) such as T-antigen, and combinations thereof. The selection and attachment of a particular targeting moiety is well within the knowledge of one of ordinary skill in the art.

The present invention also provides lyophilized pharmaceutical compositions that may be stored for long periods of time and reconstituted prior to use. In one aspect, a lyophilized pharmaceutical composition may include a lyophilized mixture of a filler excipient and a nucleic acid condensed with a cationic lipopolymer, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The lyophilized pharmaceutical composition may be in a variety of forms, ranging from dry powders to partially reconstituted mixtures.

The present invention also includes methods of making various pharmaceutical compositions containing condensed nucleic acids. In one aspect, for example, a method of making a pharmaceutical composition having a condensed nucleic acid concentrated in an isotonic solution to at least 0.5 mg/ml is provided. Such a method may include mixing a nucleic acid and a cationic lipopolymer in a filler excipient, where the cationic lipopolymer includes a cationic polymer backbone having cholesterol and polyethylene glycol covalently attached thereto, and wherein the molar ratio of cholesterol to cationic polymer backbone is within a range of from about 0.1 to about 10, and the molar ratio of polyethylene glycol to cationic polymer backbone is within a range of from about 0.1 to about 10. The mixture may be lyophilized to a powder to concentrate the nucleic acid mixture and later reconstituted with a diluent to form a solution including at least about 0.5 mg/ml condensed nucleic acid in an isotonic solution.

Generally, the composition may be obtained by mixing a nucleic acid solution with a cationic lipopolymer solution in the presence of a disaccharide sugar followed by lyophilization and reconstitution in an isotonic solution. This process is scalable, producing a few milligrams (bench scale) to several thousand milligrams (GMP scale) of the highly concentrated nucleic acid formulations with prolonged shelf life. As has been described, the cationic lipopolymer has a cationic polymer backbone to which polyethylene glycol and cholesterol are attached by covalent linkages. In the case of a polyethylenimine backbone, in one aspect the stoichiometry between polyethylene glycol and polyethylenimine and between cholesterol and polyethylenimine is in the range of 0.5-10 and 0.1-10, respectively. The chemical composition of the cationic polymer may be important to obtaining highly concentrated stable nucleic acid formulations. Cationic polymers that do not exhibit cholesterol and PEG attachment do not tend to produce stable highly concentrated formulations, as is shown in the Examples below.

The compositions according to aspects of the present invention can also be combined with other fully condensed complexes of nucleic acid to achieve greater stability of the complexes at high nucleic acid concentrations.

Aspects of the present invention also provide methods of using pharmaceutical compositions. For example, in one aspect a method of transfecting a mammalian cell may include contacting the mammalian cell with a composition as described herein, and incubating the mammalian cell under conditions to allow the composition to enter the cell and elicit biological activity of the nucleic acid. Such transfection techniques are known to those of ordinary skill in the art. Additionally, in another aspect a targeted tissue may be transfected by delivering the composition into a warm blooded organism or subject. Such targeted tissue may include any tissue or subset of tissue that would benefit from transfection. For example, and without limitation, such targeted tissue may include ovary, uterus, stomach, colon, rectum, bone, blood, intestine, pancreas, breast, head, neck, lungs, spleen, liver, kidney, brain, thyroid, prostate, urinary bladder, thyroid, skin, abdominal cavity, thoracic cavity, and combinations thereof.

### EXAMPLES

The following examples are provided to promote a more clear understanding of certain embodiments of the present invention, and are in no way meant as a limitation thereon.

### Example 1

### Preparation of Concentrated Liquid Formulations of Condensed Nucleic Acid with a Cationic Lipopolymer at a Small Manufacturing Scale

This example illustrates preparation of highly concentrated formulations of fully condensed nucleic acid at bench-scale production. This involves preparation of nucleic acid complexes with a cationic polymer followed by lyophilization and reconstitution to isotonic solutions. The nucleic acid used was a plasmid DNA encoding for IL-12 or luciferase gene, and the polymer was a polyethylenimine covalently conjugated with polyethylene glycol and cholesterol (PEG-PEI-Chol or PPC). The molar ratio between PEG and PEI and between cholesterol and PEI was 0.5-10 and 0.1-10, respectively. First, the DNA and PPC solutions were separately prepared at 5 mg/ml in water for injection and subsequently diluted to 0.15 mg/ml (DNA) and 0.554 mg/ml (PPC) at 3% lactose. The DNA in lactose solution was added to the PPC in lactose solution using a micropipette to a nitrogen to phosphate ratio (N:P ratio) of 11:1, and the formulation was incubated for 15 minutes at room temperature to allow the complexes to form. The PPC/DNA complexes in 3% lactose were lyophilized using a FREEZONE freeze dry System from LABCONCO Corp. Kansas City, Mo. 500 μl of prepared formulation was added to 2 ml borosilicate glass vials which were then lyophilized using a freeze drying program consisting of the following segments:
1) freezing segment (Ramp 0.25° C./min, hold at −34° C. for 4 hrs),
2) primary drying segment (hold at −34° C. for 24 hrs),
3) secondary drying segment (Ramp to −20° C. and hold for 24 hrs), and
4) Ramp to 4° C. at 0.25° C./min.

The resultant lyophilized powder was reconstituted with water for injection to various concentrations ranging from 0.1 mg/ml to 20 mg/ml DNA. A typical batch of small-scale preparation amounted to 100-200 mg of fully formulated DNA.

### Example 2

### Scaled Up Preparation of Concentrated Liquid Formulations of Condensed Nucleic Acid with a Cationic Lipopolymer

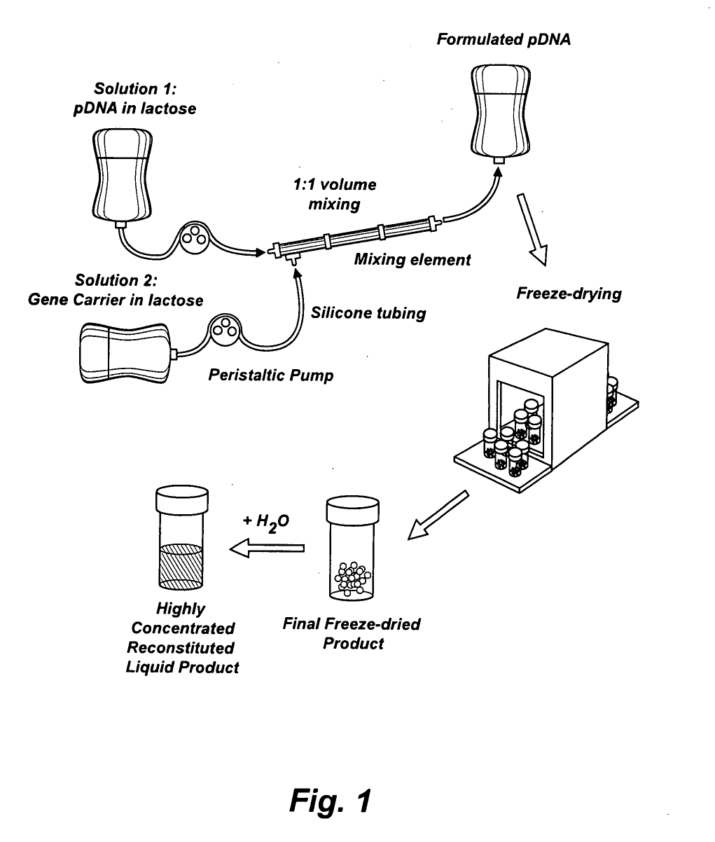

This example illustrates a scaled up preparation of highly concentrated formulations of fully condensed nucleic acid, as is shown in FIG. 1. This protocol has produced over 6000 mg of fully formulated DNA (as compared to 100-200 mg DNA produced from the small-scale preparation described in Example 1) and can be expanded to even higher production amounts. The scaled-up method involved mixing of the bulk DNA and polymer solutions with a peristaltic pump achieving an online mixing scenario to form the complexes followed by freeze-drying cycles compatible for large load. Briefly, the DNA and PPC solutions were prepared at 0.3 mg/ml and 1.1 mg/ml in 3% lactose, respectively. The two components were combined at a constant flow rate using a peristaltic pump (WATSON MARLOW, SCI 400) with a 0.89 mm internal diameter of silicon tubing (WATSON MARLOW, Z982-0088) at a flow rate of 225±25 ml/min. The two mixtures were joined by a polypropylene T-connector at the end of each tube. Mixing polymer and DNA solutions resulted in instant formation of nanoparticles. Forty milliliters of the formulated complexes were placed in 100 ml glass vials and lyophilized using a freeze-drying program consisting of the following segments:
1) pre-freeze at −50 C for up to 720 minutes,
2) primary drying at −40 C for up to 180 minutes and then at −34 C for up to 1980 minutes at 65 μm Hg, and
3) secondary drying at −25 C for up to 720 minutes, −15 C for up to 3180 minutes, −10 C for up to 1500 minutes, and 4 C for up to 1440 minutes at 65 μm Hg.

The resultant lyophilized powder was reconstituted with water for injection to various concentrations ranging from 0.1 mg/ml to 20 mg/ml DNA. A typical batch of this scale amounted to 6000 mg of fully formulated DNA.

### Example 3

### Measurement of the Particle Size of Concentrated Liquid Formulations of Condensed Nucleic Acid with a Cationic Lipopolymer

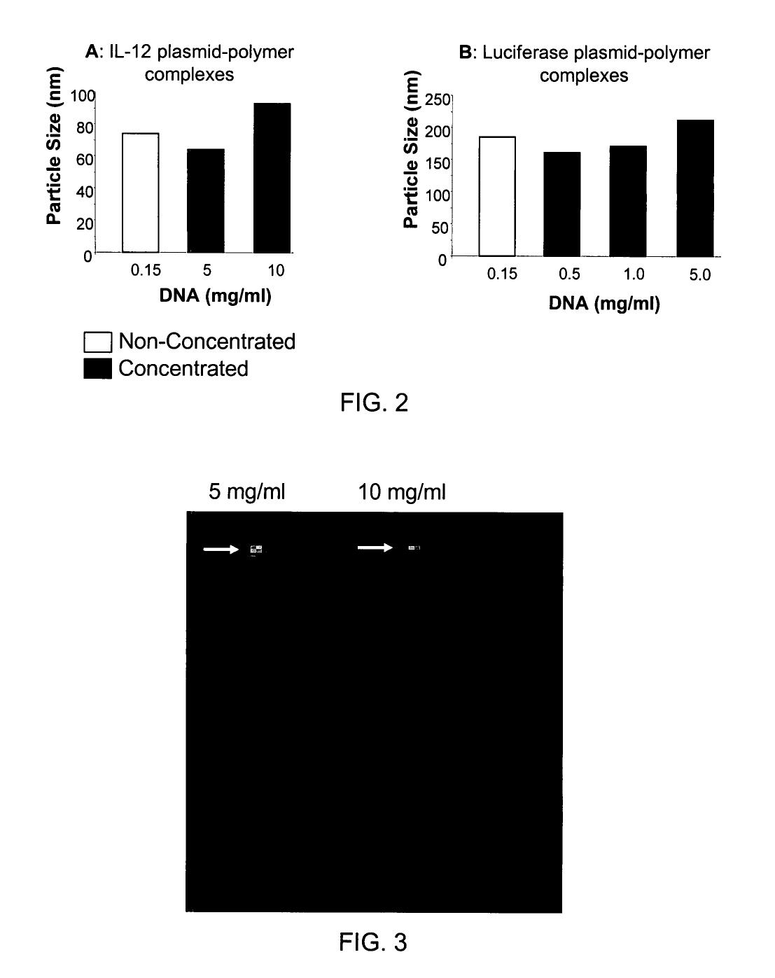

Highly concentrated formulations of plasmid DNA with cationic lipopolymer, PPC, were prepared as described in Examples 1 and 2. For particle size measurement, an aliquot of the liquid formulation was analyzed using 90Plus/BI-MAS Particle Sizer from BROOKHAVEN INSTRUMENTS Corp., Holtsville, N.Y. FIG. 2 illustrates the particle size of DNA/PPC complexes in pre-lyophilized or non-concentrated formulations (0.15 mg/ml DNA) and after reconstitution at higher concentrations ranging from 0.5 mg/ml to 10 mg/ml with IL-12 plasmid (FIG. 2A) or luciferase plasmid (FIG. 2B). Reconstitution at higher concentrations did not significantly influence the particle size, which suggests that the complexes are stable.

### Example 4

### Analysis of the Nucleic Acid Condensation of Concentrated Liquid Formulations of Nucleic Acid with a Cationic Lipopolymer

The ability of PPC polymer to condense plasmid DNA was evaluated in this example. Highly concentrated formulations of plasmid DNA with cationic lipopolymer, PPC, were prepared as described in Examples 1 and 2. The nucleic acid/polymer complexes were electrophoresed using 1% agarose gel. The electrostatic attraction of negatively charged plasmid DNA to the positively charged PPC polymer prevents the DNA from traveling through the agarose gel. As shown in FIG. 3, the DNA present in the highly concentrated formulations is fully condensed.

### Example 5

### Measurement of Nucleic Acid Concentration in Concentrated Liquid Formulation of Nucleic Acid with a Cationic Lipopolymer

The amount of nucleic acid in highly concentrated formulations of DNA and PPC complexes were quantified using an AGILENT 8453 spectrophotometer (AGILENT TECHNOLOGIES, Inc. Santa Clara, Calif.). 50 μl of formulation was diluted with 950 μl water for injection (WFI) in a quartz cuvette and absorbance was measured using 260 nm wavelength. DNA concentration was determined assuming 1 Optical density (at 260 nm)=50 μg/ml of DNA.

### Example 6

### Measurement of Transfection Activity of Concentrated Liquid Formulations of Nucleic Acid with a Cationic Lipopolymer

The transfection activity of highly concentrated formulations of DNA and PPC complexes was determined in vitro. Direct comparison was made to that of a non-concentrated formulation. Transfection complexes containing luciferase or IL-12 plasmid were prepared by methods described in Examples 1 and 2, and reconstituted at DNA concentrations ranging from 0.15 mg/ml to 10 mg/ml. Cos-1 cells (1.5×10⁵ cell/well) were seeded into 12-well tissue culture plates in 10% fetal bovine serum (FBS). Each well was incubated for 6 hours with 4 μg of complexed DNA in absence of FBS in a total volume of 500 μl of Dulbecco/Vogt Modified Eagle's Minimal Essential Medium (DMEM). When the incubation period was concluded, medium was replaced with 1 ml fresh DMEM supplemented with 10% FBS for another 40 hours. At the end of the incubation period, transfection activity was measured in the cell culture medium (IL-12) or cell lysate (luciferase). For measurement of IL-12 levels, cell culture medium was directly analyzed by an IL-12 ELISA assay. For luciferase measurement, cells were washed with phosphate-buffered saline and lysed with TENT buffer (50 mM Tris-Cl [pH8.0] 2 Mm EDTA, 150 mM NaCl, 1% Triton X-100). Luciferase activity in the cell lysate was measured as relative light units (RLU) using an Orion Microplate Luminometer (BERTHOLD DETECTION SYSTEMS, Oak Ridge, Tenn.). The final values of luciferase were reported in terms of RLU/mg total protein. The total protein level was determined using a BCA protein assay kit (PIERCE BIOTECHNOLOGY, Inc., Rockford, Ill.). The levels of IL-12 and luciferase expression from highly concentrated formulations of IL-12 and luciferase plasmid/PPC complexes are shown in FIG. 4A and FIG. 4B, respectively. The data shows transfection activity of nucleic acid complexes in highly concentrated form was preserved.

### Example 7

### Evaluating Various Excipient Sugars in the Preparation of Concentrated Liquid Formulations of Nucleic Acid with Cationic Lipopolymer and Characterization Thereof

Two commonly used sugars, lactose and sucrose, were evaluated as potential bulking or filler agents during lyophilization process for the preparation of highly concentrated formulations. PPC/DNA complex were prepared in lactose and sucrose each at 3%, 1.5% and 0.3%. Formulations were lyophilized using protocol as in Example 1. Following the freeze-drying process, formulations were reconstituted with WFI to a final DNA concentration of 0.5 mg/ml, 1 mg/ml and 5 mg/ml. Particle size and in vitro gene transfer were evaluated for these various formulation. As shown in Table 1, both particle size and transfection activity was preserved whether the cryoprotectant filler was sucrose or lactose. These results show more than one type of sugar can be used to prepare physico-chemically and biologically stable high concentrations of nucleic acid with cationic polymer.

TABLE 1
Evaluation of excipient sugars in the preparation of concentrated isotonic
formulations of nucleic acid with cationic polymer.

| | | DNA (mg/m) | | Prticle size (nm) | | Luc Expression | |
|---|---|---|---|---|---|---|---|
| Prelyo | Post- | Prelyo | | Pre- | Post- | (RLU/mg protein) | |
| ph. | Lyo | ph. | Post-Lyo | lyoph. | Lyo | Pre-lyo. | Post-Lyo |
| Lactose (w/v) | | | | | | | |
| 10.0% | N/A | 0.15 | N/A | 117.00 | N/A | 8,160,748 | |
| 3.0% | 10.0% | 0.15 | 0.50 | 123.00 | 200.00 | | 9,484,771.98 |
| 1.5% | 10.0% | 0.15 | 1.00 | 121.00 | 135.00 | | 7,492,002.47 |
| 0.3% | 10.0% | 0.15 | 5.00 | 150.00 | 209.00 | | 6,442,482.87 |
| Sucrose (w/v) | | | | | | | |
| 10.0% | N/A | 0.15 | N/A | 160.00 | N/A | 12,698,431 | |
| 3.0% | 10.0% | 0.15 | 0.50 | 137.00 | 154.00 | | 5.995,053 |
| 1.5% | 10.0% | 0.15 | 1.00 | 125.00 | 206.00 | | 8,004,970 |
| 0.3% | 10.0% | 0.15 | 5.00 | 131.00 | 244.00 | | 9,066,137 |

### Example 8

### IL-12 Expression in Normal Brain Parenchyma After Intracranial Expression of Concentrated Liquid Formulations of Nucleic Acid with Cationic Lipopolymer

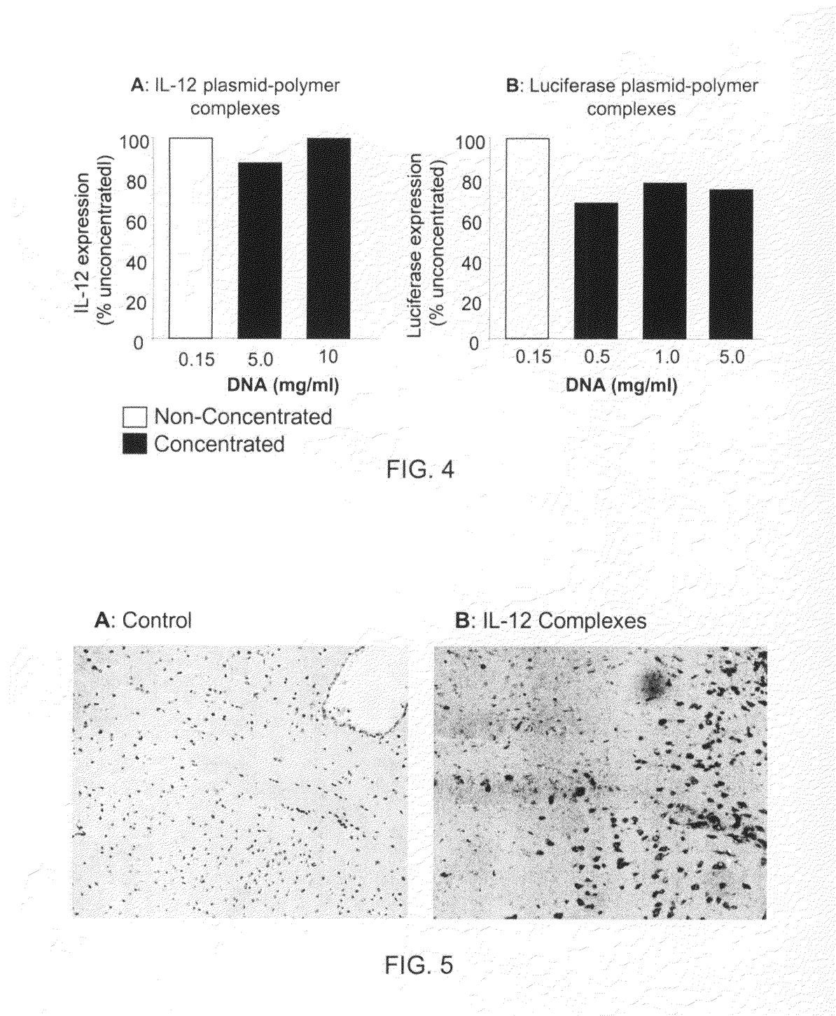

Direct administration of IL-12 plasmid with cationic polymer, PPC, in normal brain tissue was examined to determine if highly concentrated formulation of nucleic acid and cationic lipopolymer is biologically active in vivo. Immunohistochemcial staining for IL-12 was performed on slices of brains from animals euthanized 14 days or 1 month after treatment. Brain parenchyma of animals treated with PPC alone did not show any IL-12 staining (FIG. 5A). In contrast, brain parenchyma of mice injected with pmIL-12/PPC intracranially stained positive for IL-12 (FIG. 5B). This experiment demonstrates biological activity of nucleic acid complexes with a cationic polymer is preserved during the concentration process. In addition, it can be concluded that the cytokine remains present for at least a month after injection. Moreover, the presence of this cytokine in the brains of animals that remained alive until euthanized suggests that the actual expression of IL-12 does not cause lethal toxicity in brain.

### Example 9

### Efficacy of Concentrated Liquid Formulations of Nucleic Acid with Cationic Lipopolymer in a Mouse Glioma Model

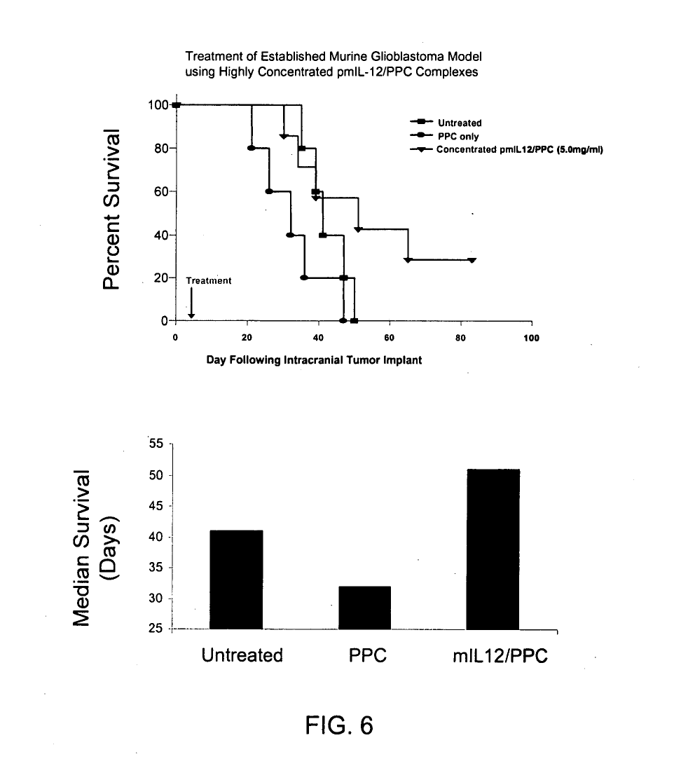

The anticancer efficacy of highly concentrated formulations of fully complexed nucleic acid expressing IL-12 gene was examined in a mouse glioma model. Tumors were implanted in the cerebral cortex of mice by intracranial injection of 1×10⁵ GL261 glioma cells together with the co-injection of 3 ul of IL-12/PPC complexes from highly concentrated formulation of 5 mg/ml IL-12 plasmid DNA. The animals were monitored for any sign of neurotoxicity and autopsied, when possible, to confirm that death was due to the intracranial tumor. Survival was plotted using a Kaplan-Meier survival analysis. A single intracranial injection of pmIL-12/PPC complexes administered at 15 ug plasmid dose was well tolerated as no significant adverse effects were observed. A single injection of pmIL-12/PPC complexes at 15 ug plasmid dose produced a significant enhancement in animal survival (FIG. 6).

### Example 10

### Biological Activity of Concentrated Liquid Formulations of Nucleic Acid with Cationic Lipopolymer in Ovarian Cancer Patients

The biological activity of highly concentrated formulation of fully condensed nucleic acid expressing IL-12 gene was examined in a patients with recurrent ovarian cancer. Four weekly intraperitoneal administrations of highly concentrated isotonic formulations of IL-12 plasmid and PPC in women with recurrent ovarian cancer produced significant levels of IFN-γ, a surrogate marker of IL-12, in peritoneal fluid of treated patients. The IFN-y levels varied from 20 to 275 pg/ml peritoneal fluid. These data demonstrates that the highly concentrated formulation of IL-12 nucleic acid is suitable for clinical application.

### Example 11

### Evaluating the Effect of Chemical Composition of Cationic Polymer on the Properties of Concentrated Liquid Formulations of Nucleic Acid with Cationic Lipopolymer

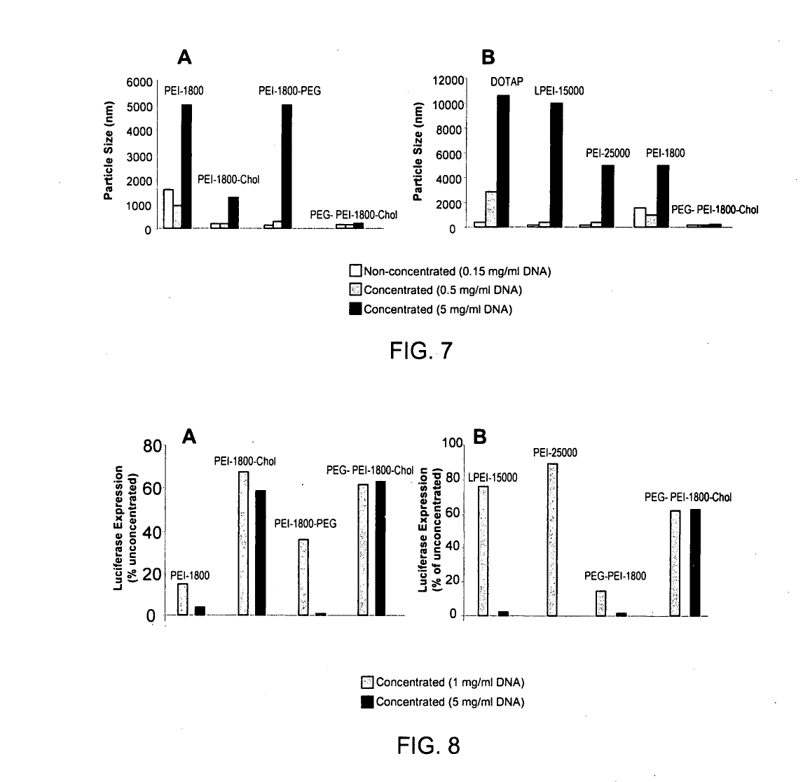

Previous attempts have demonstrated that concentrating nucleic acid formulations with cationic gene carriers such as lipid or polymers is highly challenging due to poor stability and loss of transfection as a result of the concentration process. To determine if the success in producing physico-chemically and biologically stable high concentrations of fully condensed nucleic acid is unique to the chemical composition of the test cationic polymer, PEG-PEI-Cholesterol (PPC), other cationic polymers were tested, including that of free PEI, PEI linked to cholesterol or PEI linked to PEG and a cationic liposome DOTAP. DNA complexes were prepared at 0.15 mg/ml and then concentrated to 0.5 and 5 mg/ml as described in Example 1. Particle size and transfection activity was determined as described in Example 3 & 6. As shown in FIGS. 7 & 8, DNA complexes prepared with free PEI (PEI1800, PEI15000, PEI 25000) or PEI-Cholesterol, PEI-PEG or cationic lipid DOTAP did not produce stable complexes as these complexes aggregated and lost transfection activity after lyophilization and reconstitution to 0.5 mg/ml or 5 mg/ml. The destabilizing effects were more prominent at 5 mg/ml than at 0.5 mg/ml. In comparison, DNA complexes prepared with PEG-PEI-cholesterol (PPC) maintained their physico-chemical and transfection properties during lyophilization and reconstitution at high DNA concentrations (FIGS. 7 & 8). These results suggest covalent modification of cationic polymer with cholesterol and PEG is critical to activity preservation during the concentration process.

### Example 12

### Long-Term Stability of the Lyophilized or Concentrated Liquid Formulations of Nucleic Acid with Cationic Polymer

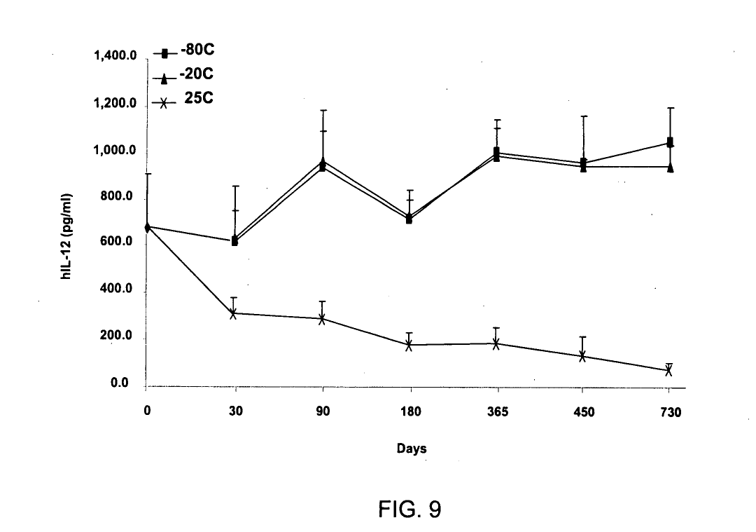

Large scale lots of lyophilized IL-12/PPC complexes were prepared under cGMP with the method outlined in Example 2 and stored at −80° C., −20° C., 4° C., and 25° C. (60% RH) for stability evaluation. At the time of analysis, vials were removed from storage and 2.4 mL of WFI was added. For each sample pH, DNA concentration, osmolality, particle size and biological activity measured. As shown in FIG. 9, the DNA concentration, pH, osmolality and particle size of the IL-12/PPC complexes were maintained during the two-year storage at the indicated temperatures. The gene transfer activity of pIL-12/PPC was quantified in COS-1 cells as described in Example 6. The COS-1 cells were transfected with the biological material at 4 μg DNA. The levels of IL-12 in cell culture media were quantified 48 hours after the transfection with a commercially available ELISA kit. The bioactivity results from the two-year stability study are illustrated in FIG. 9. There was no significant change in bioactivity of the biological product during the storage period at −80° C. or −20° C. At time 0, the activity was 151±130 pg/mL and the rest of the data fluctuates within this standard deviation, except for 25° C. where there is a consistent decline over time. At 4 C a drop in transfection activity was observed at 360 days but due to insufficient samples no follow up time points were available to reach a conclusive assessment.

### Example 13

### Stability of the Reconstituted Material of Concentrated Liquid Formulations of Nucleic Acid with Cationic Polymer

The stability of reconstituted material was examined in a separate study. Lyophilized IL-12 plasmid DNA/PPC complexes were prepared according to the method described in Example 2, and reconstituted in water for injection to 0.5 mg/ml. The reconstituted material was stored at 4° C. Samples were removed on day 60 and 90 and analyzed for particle size, osmolality, and gene expression. The lyophilized product stored in sealed vials at −80° C. was analyzed simultaneously for comparison. As shown in Table 2, the reconstituted EGEN-001 is stable at 4° C. for at least 90 days after reconstitution with WFI. None of the stability parameters including DNA concentration, particle size, osmolality or gene expression was significantly altered when compared to the lyophilized material stored in sealed vials at −80° C.

TABLE 2
Long-term stability of the reconstituted form of highly concentrated
and fully condensed isotonic formulations of nucleic acid with
cationic polymer at 4 C.

| Stability | Days | | | | | |
|---|---|---|---|---|---|---|
| Parameters | 0 | 60 | 90 | 180 | 270 | 365 |
| Particle size | 102 | 98 | 101 | 97 | 103 | 98 |
| (nm) | | | | | | |
| Osmolarity | 303 | 309 | 303 | 312 | 312 | 306 |
| (mOsmole) | | | | | | |
| pH | 2.75 | 2.66 | 2.69 | 2.7 | 2.73 | 2.59 |
| DNA (mg/ml) | 0.49 | 0.49 | 0.50 | 0.47 | 0.50 | 0.50 |
| IIL-12 | 1220 | 1681 | 1164 | 1062 | 1409 | 476.3 |
| Expression | | | | | | |
| (pg/ml) | | | | | | |

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A pharmaceutical composition, comprising:
at least about 0.5 mg/ml concentration of a nucleic acid condensed with a cationic lipopolymer suspended in an isotonic solution, said cationic lipopolymer including a cationic polymer backbone having cholesterol and polyethylene glycol covalently attached thereto, wherein a molar ratio of cholesterol to cationic polymer backbone is within a range of from about 0.1 to about 10, and a molar ratio of polyethylene glycol to cationic polymer backbone is within a range of from about 0.1 to about 10; and
a filler excipient.

2. The composition of claim 1, wherein the nucleic acid is at least about 30% condensed.

3. The composition of claim 1, wherein the nucleic acid is at least about 70% condensed.

4. The composition of claim 1, wherein the nucleic acid is at least about 90% condensed.

5. The composition of claim 1, wherein the cationic polymer backbone is a member selected from the group consisting of polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, and combinations thereof.

6. The composition of claim 5, wherein the cationic polymer backbone is polyethylenimine.

7. The composition of claim 1, wherein the concentration of the nucleic acid is at least 1 mg/ml.

8. The composition of claim 1, wherein the concentration of the nucleic acid is at least 3 mg/ml.

9. The composition of claim 1, wherein the concentration of the nucleic acid is at least 5 mg/ml.

10. The composition of claim 1, wherein the concentration of the nucleic acid is at least 10 mg/ml.

11. The composition of claim 1, wherein the ratio of amine nitrogen in the cationic polymer backbone to phosphate in the nucleic acid is from about 0.1:1 to about 100:1.

12. The composition of claim 1, wherein the ratio of amine nitrogen in the cationic polymer backbone to phosphate in the nucleic acid is from about 3:1 to about 20:1.

13. The composition of claim 1, wherein the nucleic acid is a plasmid encoding for a peptide.

14. The composition of claim 13, wherein the nucleic acid is a plasmid encoding for a member selected from the group consisting of interleukin-2, interleukin-4, interleukin-7, interleukin-12, interleukin-15, interferon-α, interferon-β, interferon-y, colony stimulating factor, granulocyte-macrophage colony stimulating factor, angiogenic agents, clotting factors, hypoglycemic agents, apoptosis factors, anti-angiogenic agents, thymidine kinase, p53, IP10, p16, TNF-α, Fas-ligand, tumor antigens, neuropeptides, viral antigens, bacterial antigens, and combinations thereof.

15. The composition of claim 13, wherein the nucleic acid is a plasmid encoding for interleukin-12.

16. The composition of claim 1, wherein the nucleic acid is a plasmid encoding for an inhibitory ribonucleic acid.

17. The composition of claim 1, wherein the nucleic acid is a synthetic short interfering ribonucleic acid.

18. The composition of claim 1, wherein the nucleic acid is an anti-sense molecule designed to inhibit expression of a therapeutic peptide.

19. The composition of claim 1, wherein the cationic polymer backbone has a molecular weight of from about 100 to about 500,000 Daltons.

20. The composition of claim 1, wherein the polyethylene glycol has molecular weight of from about 50 to about 20,000 Daltons.

21. The composition of claim 1, wherein the cationic lipopolymer further includes a targeting moiety covalently attached to either the cationic lipopolymer or to the polyethylene glycol molecule.

22. The composition of claim 21, wherein the targeting moiety is a member selected from the group consisting of transferrin, asialoglycoprotein, antibodies, antibody fragments, low density lipoproteins, cell receptors, growth factor receptors, cytokine receptors, folate, transferrin, insulin, asialoorosomucoid, mannose-6-phosphate, mannose, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents, lysosomotrophic agents, nucleus localization signals (NLS), and combinations thereof.

23. The composition of claim 1, wherein a molar ratio of polyethylene glycol to cationic polymer backbone in the cationic lipopolymer is within a range of from about 1 to about 0.10.

24. The composition of claim 1, wherein a molar ratio of polyethylene glycol to cationic polymer backbone in the cationic lipopolymer is within a range of from about 1 to about 5.

25. The composition of claim 1, wherein a molar ratio of cholesterol to cationic polymer backbone in the cationic lipopolymer is within a range of from about 0.3 to about 5.

26. The composition of claim 1, wherein a molar ratio of cholesterol to cationic polymer backbone in the cationic lipopolymer is within a range of from about 0.4 to about 1.5.

27. The composition of claim 1, wherein the filler excipient is a member selected from the group consisting of sugars, sugar alcohols, starches, celluloses, and combinations thereof.

28. The composition of claim 1, wherein the filler excipient is a member selected from the group consisting of lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, polyethylene glycol, and combinations thereof.

29. The composition of claim 1, wherein the filler excipient is a member selected from the group consisting of lactose, sucrose, trehalose, dextrose, galactose, mannitol, maltitol, maltose, sorbitol, xylitol, mannose, glucose, fructose, polyvinyl pyrrolidone, glycine, maltodextrin, and combinations thereof.

30. The composition of claim 1, wherein the filler excipient is sucrose.

31. The composition of claim 1, wherein the filler excipient is lactose.

32. The composition of claim 1, wherein the nucleic acid is a plasmid encoding for interleukin-12 gene, and the cationic polymeric backbone is polyethylenimine.

33. The composition of claim 1, wherein the at least one cholesterol molecule and the at least one polyethylene glycol molecule are independently covalently attached to the cationic polymer backbone.

34. A method of making a pharmaceutical composition having a condensed nucleic acid concentrated in an isotonic solution to at least 0.5 mg/ml, comprising:
mixing a nucleic acid and a cationic lipopolymer in a filler excipient, said cationic lipopolymer including a cationic polymer backbone having cholesterol and polyethylene glycol covalently attached thereto, wherein a molar ratio of cholesterol to cationic polymer backbone is within a range of from about 0.1 to about 10, and a molar ratio of polyethylene glycol to cationic polymer backbone is within a range of from about 0.1 to about 10;
lyophilizing the mixture to a powder; and
reconstituting the powder with a diluent to form a solution including at least about 0.5 mg/ml condensed nucleic acid in an isotonic solution.

35. A lyophilized pharmaceutical composition, comprising:
a lyophilized mixture of a filler excipient and a nucleic acid condensed with a cationic lipopolymer, said cationic lipopolymer including a cationic polymer backbone having at least one cholesterol molecule and at least one polyethylene glycol molecule covalently attached thereto.

36. The composition of claim 35, wherein the lyophilized mixture is in a powder form.

37. The composition of claim 1, further comprising a secondary nucleotide condensing system.

38. The composition of claim 37, wherein the secondary nucleotide condensing system includes a member selected from the group consisting of cationic lipids, cationic peptides, cyclodextrins, cationized gelatin, dendrimers, chitosan, and combinations thereof.

39. A method of transfecting a mammalian cell, comprising:
contacting the mammalian cell with the composition of claim 1; and
incubating the mammalian cell under conditions to allow the composition of claim 1 to enter the cell and elicit biological activity of the nucleic acid.

40. A method of transfecting a targeted tissue, comprising delivering the composition of claim 1 into a warm blooded organism.

41. The method of claim 40, wherein delivering the composition may further include a form of administration selected from the group consisting of intratumoral, intraperitoneal, intravenous, intra-arterial, intratracheal, intrahepaticportal, oral, intracranial, intramuscular, intraarticular and combinations thereof.

42. The method of claim 40, wherein the targeted tissue is localized in a member selected from the group consisting of ovary, uterus, stomach, colon, rectum, bone, blood, intestine, pancreas, breast, head, neck, lungs, spleen, liver, kidney, brain, thyroid, prostate, urinary bladder, thyroid, skin, abdominal cavity, thoracic cavity, and combinations thereof.
